(19) United States
(12) Patent Application Publication
(10) Pub. No.: US 2026/0112038 A1
(43) Pub. Date: Apr. 23, 2026

(54) METHODS AND RELATED ASPECTS FOR ANTIBIOTIC SUSCEPTIBILITY TESTING

(71) Applicant: ARIZONA BOARD OF REGENTS ON BEHALF OF ARIZONA STATE UNIVERSITY, Scottsdale, AZ (US)

(72) Inventors: Shaopeng Wang, Chandler, AZ (US); Brandyn Braswell, Tempe, AZ (US); Jiapei Jiang, Tempe, AZ (US); Shelley Haydel, Mesa, AZ (US)

(73) Assignee: ARIZONA BOARD OF REGENTS ON BEHALF OF ARIZONA STATE UNIVERSITY, Scottsdale, AZ (US)

(21) Appl. No.: 19/366,715

(22) Filed: Oct. 23, 2025

Related U.S. Application Data
(60) Provisional application No. 63/710,702, filed on Oct. 23, 2024.

(51) Int. Cl. G06T 7/00; C12Q 1/18; G06V 10/774; G06V 20/69

(52) U.S. Cl. CPC G06T 7/0016; C12Q 1/18; G06V 10/774; G06V 20/698; G06T 2207/10016; G06T 2207/10056; G06T 2207/20081; G06T 2207/30024; G06T 2207/30072

(57) ABSTRACT

Provided herein are methods of determining antibiotic susceptibility of bacteria. In some embodiments, the methods include obtaining a series of images from partitioned samples over a length of time using a large volume scattering imaging (LVSi) technique to produce a set of image data. In some embodiments, the methods include extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features, and passing the set of extracted phenotypic features through a trained machine learning model that predicts the identity and the antibiotic susceptibility of the bacterial cells in the partitioned samples. Related kits, devices, systems, computer readable media, and additional methods are also provided.

### CROSS-REFERENCE TO RELATED APPLICATIONS

This application claims priority to, and the benefit of, U.S. Provisional Patent Application Ser. No. 63/710,702, filed Oct. 23, 2024, the disclosure of which is incorporated herein by reference.

### STATEMENT REGARDING FEDERALLY SPONSORED RESEARCH OR DEVELOPMENT

This invention was made with government support under R01 AI138993 awarded by the National Institutes of Health. The government has certain rights in the invention.

### FIELD

This disclosure relates generally to medical applications, such as pathology.

### BACKGROUND

Urinary tract infections (UTIs) are a common type of bacterial infection, affecting half of the US population during their lifetime. Although many UTIs are uncomplicated, mistreatment of these infections can develop into more serious infections such as sepsis. With the rise of antimicrobial resistance the ability to effectively treat UTIs prior to becoming a more serious infection is becoming increasingly more difficult. Therefore, rapid UTI pathogen identification (ID) and antimicrobial susceptibility testing (AST) are needed for timely treatment of UTIs with the correct antibiotics. However, the gold standard methods for UTI pathogen screening and phenotypic AST are culture-based, which takes 48 h or more to produce results. The culture of the samples is necessary for these methods to isolate and enrich the pathogens, so optical density signals can increase to reach traditional levels of detection.

Accordingly, there is a need for additional methods for use in infectious disease detection.

### SUMMARY

The present disclosure relates, in certain aspects, to a rapid pathogen identification (ID) and antimicrobial susceptibility testing (AST) technology that provides timely diagnosis of resistant infections and delivery of accurate antibiotic treatment at primary health-care settings, including hospitals and point-of-care (POC). In some embodiments, the present disclosure provides a point-of-care AST (POCAST™) technology based on a large-image-volume scattering imaging (LVSi) technique that enables direct detection of individual bacterial cells in clinical samples without culturing or pathogen isolation, and a machine-learning model that allows fast detection of pathogen and determination of antimicrobial susceptibility. Although the technology disclosed herein is suitable for diagnosing various types of infections, for clarity of illustration this disclosure frequently emphasizes urinary tract infections (UTIs) as an exemplary application of the technology. UTIs affect millions of people annually, and the pathogens that usually cause UTIs are the organisms that pose the highest of antimicrobial resistance, including carbapenem-resistant Enterobacteriaceae (CRE) and extended spectrum β-lactamase (ESBL)-producing Enterobacteriaceae. These and other aspects will be apparent upon a complete review of the present disclosure, including the accompanying figures.

According to various embodiments, a method of determining antibiotic susceptibility of bacteria is presented. The method includes: obtaining a series of images from partitioned samples over a length of time using a large volume scattering imaging (LVSi) technique to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and, outputting the set of predicted identity and antibiotic susceptibility data, thereby determining the antibiotic susceptibility of the bacteria. In some embodiments, the passing step is optional.

Various optional features of the above embodiments include the following. The obtaining step is performed in the absence of culture-based isolation and/or enrichment of the partitioned samples. The method comprises substantially removing non-bacterial particles and non-bacterial cells from the partitioned samples prior to performing the obtaining step. The partitioned samples comprise between about 103 bacterial cells/mL and about 108 bacterial cells/mL. The partitioned samples comprise urine. The partitioned samples are disposed in a microplate. The multiple phenotypic features and/or the phenotypic feature changes are selected from the group consisting of: a cell count, a cellular shape, a cell division, a cellular motion, a cellular physiology, a cell size, and a cellular morphology. The cellular shape is analyzed via detected bacterial cell intensity changes over time. The multiple phenotypic features comprise 3, 4, 5, 6, 7, 8, 9, 10, or more phenotypic features. The set of image data comprises LVSi videos. The method further comprises administering an antibiotic to treat a bacterial infection in a test subject based at least in part on the set of predicted identity and antibiotic susceptibility data. The method comprises determining minimum inhibition concentrations (MIC) of the antibiotics. The bacteria comprise one or more of Escherichia coli, Klebsiella pneumoniae, Staphylococcus saprophyticus, or Enterococcus faecalis. A kit for performing the method. A point-of-care device for performing the method. The method comprises obtaining the partitioned samples from one or more test subjects. The method comprises outputting the set of predicted identity and antibiotic susceptibility data within about 3 hours of obtaining the partitioned samples from one or more test subjects. The test subjects are suspected of having urinary tract infections (UTIs).

According to various embodiments, a method of determining antibiotic susceptibility of bacteria is presented. The method includes obtaining a series of images from partitioned samples over a length of time using a large volume scattering imaging (LVSi) technique to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; producing a set of predicted identity and antibiotic susceptibility data from the set of extracted phenotypic features; and outputting the set of predicted identity and antibiotic susceptibility data, thereby determining the antibiotic susceptibility of the bacteria. In some embodiments, the extracting and/or producing steps comprise using only bacterial cell intensities and counts to produce the set of extracted phenotypic features and/or the set of predicted identity and antibiotic susceptibility data.

According to various embodiments, a system for determining antibiotic susceptibility of bacteria is presented. The system includes a large volume scattering imaging (LVSi) apparatus configured to receive partitioned samples; and, a controller operably connected to the LVSi apparatus, which controller comprises, or is capable of accessing, computer readable media comprising non-transitory computer-executable instructions which, when executed by at least one electronic processor, perform at least: obtaining a series of images from partitioned samples over a length of time using LVSi apparatus to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and, outputting the set of predicted identity and antibiotic susceptibility data. In some embodiments, the set of image data comprises LVSi videos.

According to various embodiments, a computer readable media is presented. The computer readable media comprises non-transitory computer executable instructions which, when executed by at least one electronic processor, perform at least: obtaining a series of images from partitioned samples over a length of time using a large volume scattering imaging (LVSi) technique to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and, outputting the set of predicted identity and antibiotic susceptibility data.

### DRAWINGS

The above and/or other aspects and advantages will become more apparent and more readily appreciated from the following detailed description of examples, taken in conjunction with the accompanying drawings, in which:

FIG. 1 is a flowchart that shows an exemplary method of determining antibiotic susceptibility of bacteria in accordance with an embodiment.

FIG. 2 is a schematic diagram of an exemplary system suitable for use with certain aspects disclosed herein.

FIG. 3 shows exemplary features of a large volume scattering imaging technique to capture multiple phenotypic features of single bacterial cells at clinically relevant pathogen concentrations and determine infection status and AST without culture isolation and sample enrichment in accordance with an embodiment.

FIG. 4 schematically depicts various aspects of a POCAST™ workflow in which the total time (from sample collection to ID/AST report) is <3 hours in accordance with an embodiment.

FIGS. 5A-5C shows aspects of an exemplary high throughput Large Volume Scattering imaging (LVSi) system consisting of a pair of red LED light (780 nm near infrared LED light) and optics to illuminate a large volume of the sample, and a wide-view and deep focal depth imaging system to collect back scattered light and form a low noise video of the individual bacterial cells at clinically relevant concentrations. (A) Schematic of the microplate-based LVSi system that can measure 96 samples simultaneously; (B) A picture of the preliminary microplate-based LVSi testing setup; (C) A typical back scattering LVSi image, where the inset is a magnified view of the boxed region.

FIG. 6 schematically depicts an exemplary machine learning model that recognizes each particle (bacterial cell or another substance) in the urine sample in a LVSi video, extracts multiple phenotypic features of each particle over time (t), feeds the features into a neural network (e.g., Long Short-Term Memory Model) as an input, trains the model using LVSi videos and patient samples with ground truth results obtained with the gold standard methods, and provides an output (ID and AST).

FIGS. 7A-7D are plots showing the detection of bacteria by LVSi particle tracking. (A) Single cell motion and intensity mapping for E. coli and 1 μm polystyrene beads. (B) Comparison of the corresponding micro motion (top panel) and intensity fluctuation (lower panel) of single E. coli cell and 1 μm polystyrene bead. (C) Object (particle) intensity changes of E. coli over 90 min growth. (D) Count (division events) changes of E. coli over 90 min growth.

FIGS. 8A-8D are plots showing the detection of UTI pathogens by machine learning [support vector machine (SVM)]. (A) Detect E. coli (UPEC) from urine samples containing non-bacterial particles. (B) Differentiate E. coli vs S. saprophyticus. (C) Rapid (10 min sample to result) detection of positive UTI infections in 104 clinical urine samples show high agreement with clinical reported results. (D) The ROC curve for UTI diagnosis by LVSi. The sensitivity and specificity are 84% and 100%, respectively.

FIGS. 9A-9F shows the determination of MIC from multi-phenotypic features. Motion changes. (A) LVSi reveals tracks (bright lines) of E. coli O157: H7 cells for 10 seconds, where the track lengths reflect the distances moved by the individual cells. Track images from left to right correspond to low to high polymyxin B (PMB) concentrations, and shortening of the track lengths at high PMB concentrations indicate motion slowing. Scale bars: 150 μm. (B) Dose response curve (mean displacement of all bacterial tracks vs. PMB dose). Morphology changes. (C) Bright field and LVSi (upper-right inset) images of E. coli XL1-B culture after 90 min exposure to ampicillin at different concentrations. The LVSi image intensity increases, due to cell elongation, at low ampicillin concentrations, and decreases, due to cell lysis, at high concentrations. Scale bars of LVSi: 50 μm; scale bars of bright field images: 10 μm. (D) Dose response (mean intensity of bacterial cells vs. ampicillin dose). Growth (division) rate changes. (E) LVSi images of uropathogenic E. coli after 90 min exposure to increasing concentrations of ciprofloxacin (from left to right), showing decreasing number of the bacterial cells. (F) Cell number vs. ciprofloxacin concentration. Scale bars of LVSi: 50 μm. Error bars in (B) and (D) represent standard error of the mean (SEM), and error bars in (F) represent standard deviation.

FIGS. 10A-10C are plots showing direct LVSi-AST with infection positive clinical urine samples in 90 minutes. The LVSi total particle intensity change (normalized to the intensity of control sample at 90 min) over 90 min of (A) 38 susceptible samples and (B) 17 resistant samples in the presence of 2 μg/mL ciprofloxacin. Open grey circles/squares are individual sample values, while filled black dots and open grey squares represent mean values. (C) Comparison of reference method (BD Phoenix) and POCAST for susceptibility determination at 90 min. LVSi responses to antibiotics is defined as ratio of the total particle scattering intensity change between antibiotic-exposed and -unexposed samples (control).

FIGS. 11A and 11B show an LVSi image quality comparison between (A) cuvette-based forward scattering image, and (B) microplate-based backward scattering image. Both images measure 105 CFU/mL E. coli ATCC 25922, recorded at 2× optical zoom. The insets show the plots of line profiles of typical bacterial images marked by the arrows in the images. The signal-to-noise ratio (SNR) are calculated as ratio of the peak particle image intensity and the standard deviation of the background intensity fluctuation.

FIG. 12 show high-throughput LVSi AST results for E. coli (ATCC 25922) with four common UTI antibiotics: ampicillin, nitrofurantoin, ciprofloxacin, and cefazolin tested in a 96-well microplate in a single experiment. Each drug is measured at 8 different doses (including 0 μg/mL as a growth control) with 3 replicates. Each well is imaged sequentially for 9 sec at 10 fps. Subsequent imaging occurs every 20 min. The heatmap shows the normalized light intensity changes of E. coli over a 140-minute period in response to varying concentrations of antibiotics. Each row corresponds to a specific antibiotic and its concentration, while each column represents a different time point during the imaging process. The MIC concentration determined by concurrent standard CFU assays are marked with an asterisk (*). The gray boxes highlight the LVSi-determined MIC concentrations defined when LVSi intensity stopped increasing before the end of the assay, which is equal to or within one dilution factor of the CFU MIC values.

FIG. 13 is a functional diagram of POCAST™ instrument and software in accordance with an embodiment.

FIG. 14 shows a POCAST™ microplate design in accordance with an embodiment.

FIG. 15. Mycobacterium abscessus smooth fold-change growth response to antibiotics. Growth trajectories are shown for Amikacin, Bedaquiline, Clarithromycin, and Clofazamine across different concentrations, plotted as the mean fold-change in particle count (n=3 wells per dose). Shaded regions represent the interquartile range (IQR). The solid black line denotes the determined MIC (based on OD readings). The green and red lines correspond to the positive and negative controls, respectively. Non-MIC concentrations are dimmed for clarity. Imaging occurred over a 24-hour period in 3-hour intervals.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Classifier. As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Sample: As used herein, a “sample,” such as a biological sample, is a sample obtained from a subject. As used herein, biological samples include all clinical samples including, but not limited to, cells, tissues, and bodily fluids, such as saliva, tears, breath, and blood; derivatives and fractions of blood, such as filtrates, dried blood spots, serum, and plasma; extracted galls; biopsied or surgically removed tissue, including tissues that are, for example, unfixed, frozen, fixed in formalin and/or embedded in paraffin; milk; skin scrapes; nails, skin, hair; surface washings; urine; sputum; bile; bronchoalveolar fluid; pleural fluid, peritoneal fluid; cerebrospinal fluid; prostate fluid; pus; or bone marrow. In a particular example, a sample includes blood obtained from a subject, such as whole blood or serum. In another example, a sample includes cells collected using an oral rinse. The sample may be isolated from the subject and then directly utilized in a method for determining the presence or absence of antibodies, or alternatively, the sample may be isolated and then stored (e.g., frozen) for a period of time before being subjected to analysis.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

System: As used herein, “system” in the context of analytical instrumentation refers a group of objects and/or devices that form a network for performing a desired objective.

Treat: As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

Value: As used herein, “value” generally refers to an entry in a data set that can be anything that characterizes the feature to which the value refers. This includes, without limitation, numbers, words or phrases, symbols (e.g., +or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. Introduction

In some aspects, the present disclosure describes a culture-independent technology for point-of-care diagnosis of antimicrobial-resistant bacteria in urinary tract infections, among other types of infections, within 3 hours, by imaging urine samples or other sample types directly with a large-image-volume imaging technique and analyzing the data with a machine-learning model. Among other attributes, the technology provides for precise antibiotic prescriptions and accurate treatment of the patient on the same day of visit for UTI and a core technology for addressing a broader range of bacterial infections. These and other aspects will be apparent upon a complete review of the present disclosure, including the accompanying figures.

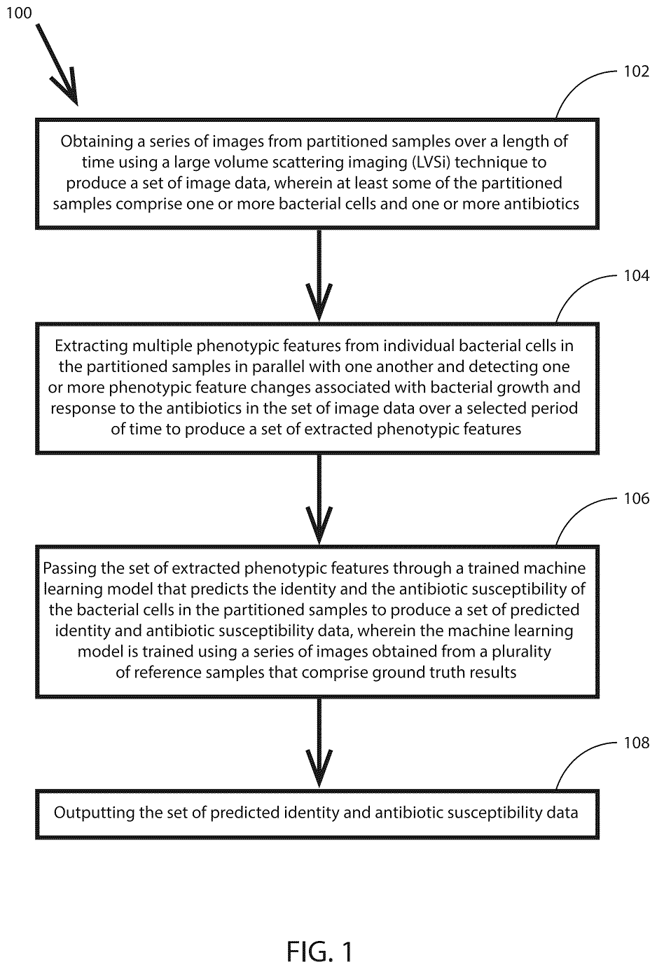

To illustrate, FIG. 1 is a flowchart that shows an exemplary method of determining antibiotic susceptibility of bacteria in accordance with an embodiment. As shown, method 100 includes obtaining a series of images from partitioned samples (e.g., disposed in a microplate or other sample container) over a length of time using a large volume scattering imaging (LVSi) technique to produce a set of image data, wherein at least some of the partitioned samples comprise one or more bacterial cells and one or more antibiotics (step 102). Method 100 also includes extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features (step 104). In addition, method 100 also includes passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results (step 106) and outputting the set of predicted identity and antibiotic susceptibility data (step 108).

In some embodiments, the obtaining step (step 102) of method 100 is performed in the absence of culture-based isolation and/or enrichment of the partitioned samples. In some embodiments, method 100 includes substantially removing non-bacterial particles and non-bacterial cells from the partitioned samples prior to performing the obtaining step (step 102). In some embodiments, the partitioned samples comprise between about 103 bacterial cells/mL and about 108 bacterial cells/mL. In some embodiments, the partitioned samples comprise urine. In some embodiments, the partitioned samples are disposed in a microplate.

In some embodiments, the multiple phenotypic features and/or the phenotypic feature changes are selected from, for example, a cell count, a cellular shape, a cell division, a cellular motion, a cellular physiology, a cell size, and a cellular morphology. In some embodiments, the multiple phenotypic features comprise 3, 4, 5, 6, 7, 8, 9, 10, or more phenotypic features. In some embodiments, the set of image data comprises LVSi videos. In some embodiments, method 100 further includes administering an antibiotic to treat a bacterial infection in a test subject based at least in part on the set of predicted identity and antibiotic susceptibility data. In some embodiments, method 100 includes determining minimum inhibition concentrations (MIC) of the antibiotics. In some embodiments, the bacteria comprise one or more of Escherichia coli, Klebsiella pneumoniae, Staphylococcus saprophyticus, or Enterococcus faecalis, although essentially any bacteria can be analyzed as described herein. In some embodiments, kits are provided for performing the methods of the present disclosure. In some embodiments, point-of-care devices for performing the methods described herein are provided.

In some embodiments, method 100 includes obtaining the partitioned samples from one or more test subjects. In some embodiments, method 100 includes outputting the set of predicted identity and antibiotic susceptibility data within about 3 hours of obtaining the partitioned samples from one or more test subjects. In some embodiments, the test subjects are suspected of having urinary tract infections (UTIs).

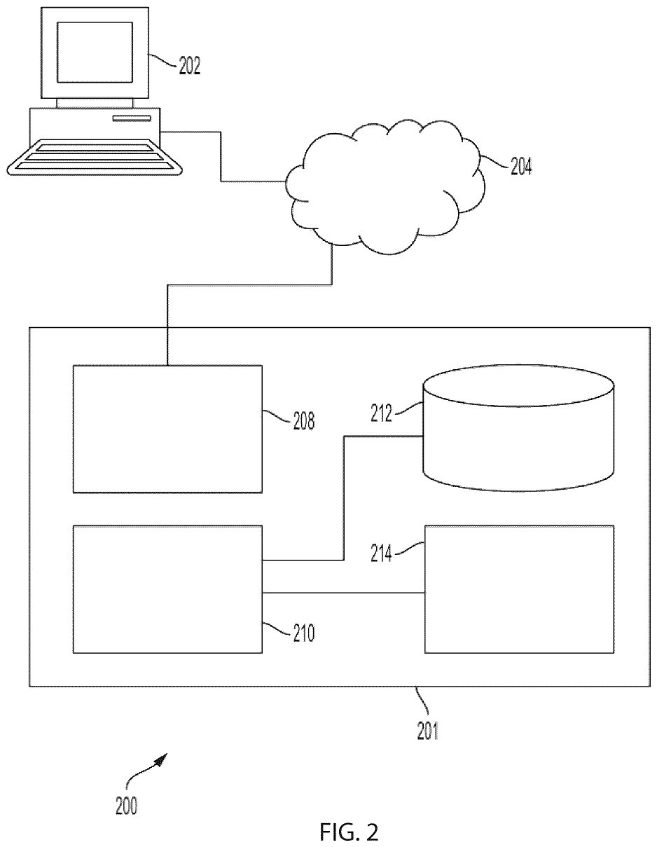

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of data suitable for use as a training corpus as disclosed herein. According to some embodiments, each component is implemented as a vector, such as a feature vector.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. Description of Example Embodiments

### Example 1: Microplate-Based High Throughput Large Volume Scattering Imaging System (HT-LVSi) for Rapid Antimicrobial Susceptibility Test of Bacteria in Liquid Samples

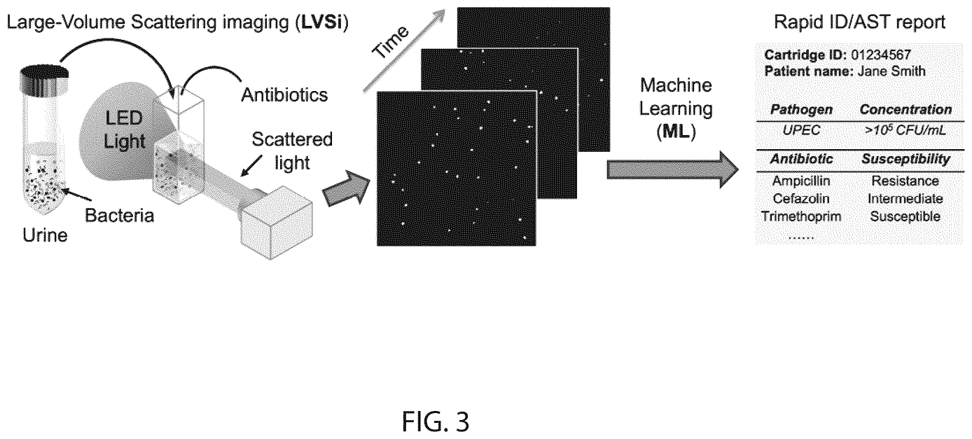

To address challenges posed by infectious disease detection and antimicrobial susceptibility testing, a large volume scattering imaging (LVSi)-based technique is presented, featuring large image volume to capture enough bacterial cells in low-pathogen-concentration patient urine samples, without requiring culture-based isolation and enrichment, and provide accurate imaging and tracking of each individual bacterial cell in the urine samples. Software algorithms have been developed to extract multiple phenotypic features (e.g., count, shape, division, and motion) of each bacterial cell, and to detect the phenotypic feature changes associated with bacteria growth and response to antibiotics. In this way, we can rapidly detect active infections and determine minimum inhibition concentrations (MIC) of tested antibiotics (FIG. 3). In some embodiments, the LVSi technology of the present disclosure provides for rapid UTI pathogen detection and AST (from loading raw patient urine sample to results) in 30-120 min. In some embodiments, the present disclosure provides a point-of-care AST (POCAST™) system for UTI pathogen detection and simultaneous AST of common UTI antibiotics in 3 hours, using a high-throughput and low-cost design. In some embodiments, for example, the methods disclosed herein provide for the simultaneous detection and multi-drug AST of common UTI bacteria in 104-108 CFU/mL in a single test within 3 hours (e.g., within 3 hours from raw sample loading to results), which enables precise antibiotic prescription on the same day of patient visit. In some embodiments, the methods disclosed herein also provide accurate UTI pathogen detection (Specificity: 90% Sensitivity: 90%) and AST (Category Agreement: 89.9%; Major Discrepancy: 3%; and Very Major Discrepancy: 1.5%).

By way of additional background, the misuse and overuse of the broad-spectrum antibiotics has led to widespread development of antimicrobial resistance, posing long-term threats to public health. Multidrug-resistant bacteria persist in many healthcare settings, leading to a wide range of acute and nosocomial infections with high mortality rates. Each year, resistant infections cause over 2.8 million infections and over 35,000 deaths in the US alone. To prescribe a patient with effective antibiotics, a key step is to perform pathogen identification (ID) and antimicrobial susceptibility testing (AST). Currently, ID/AST is performed in microbiology labs using established clinical protocols, which take several days. This slow ID/AST leads to increased patient mortality and poor clinical outcomes, forcing the use of broad-spectrum antibiotics. A faster ID/AST technology is needed to enable narrow-spectrum antibiotic administration at the earliest possible treatment stage. For such point-of-care applications, the desirable total ID/AST time, from patient sample collection to results, should be less than 3 hours to allow precise treatment of the infection on the same day of patient visit.

In some aspects, the methods and other aspects of the present disclosure utilize Large-image-Volume Scattering imaging (LVSi) techniques, featuring large image volume and low noise, to detect single bacterial cells in urine samples at clinically-relevant, low bacterial concentrations (e.g., 104-105 CFU/mL) without culturing and sample enrichment, and to extract multiple phenotypic features of single bacterial cells precisely for rapid ID and AST (FIG. 3). In some aspects, the methods and other aspects of the present disclosure also utilize machine-learning (ML) assisted image processing software capable of analyzing the vast datasets extracted from the LVSi videos, including multiple phenotypic features from single bacterial cells and other substances in the urine sample, as well as phenotypic feature changes over time. We have carried out substantial studies, including using raw clinical samples, to demonstrate the capabilities of the proposed POCAST™ for rapid pathogen ID and AST.

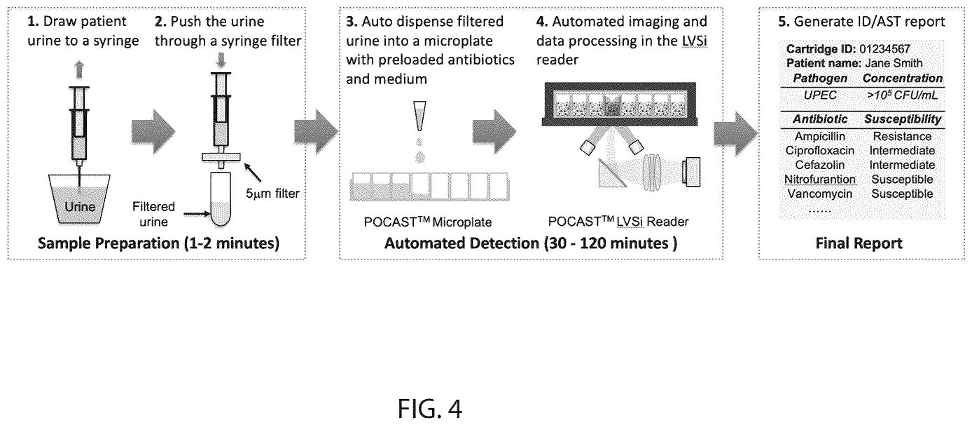

An exemplary benefit of the POCAST™ system disclosed herein is that it can be readily applied to address a broad range of bacterial infections. However, some embodiments specifically focus on urinary tract infections (UTIs), the most frequent bacterial infection in the outpatient setting. UTIs affect approximately 50% of women during their lifetime, with an estimated infection of 13.5 million people in the US each year and account for $3.5 billion in healthcare and societal costs. The most serious and urgent UTI threats include carbapenem-resistant Enterobacteriaceae (CRE) and extended spectrum β-lactamase (ESBL)-producing Enterobacteriaceae. In some embodiments, the workflow of POCAST™ consists of five steps (FIG. 4): 1) Draw patient urine sample into a disposable syringe. 2) Remove large particles and human cells in the sample via 5 μm syringe filtration. 3) Automated dispense of the filtered urine into a sample microplate that has preloaded antibiotics and medium. 4) Load the microplate into the POCAST™ reader, enter sample information (plate ID is registered automatically with barcode), and start the test. 5) Obtain ID/AST report within 3 hours.

Current ID/AST methods fall into two categories: genotypic- and phenotypic-based methods. The former detects antibiotic resistance genes or genetic mutations conferring resistance, which limit the application to known resistance genes or specific genetic mutations. For these reasons, AST technologies based on tracking phenotypic features (e.g., bacterial physiology, size, length, number, and morphology) have been pursued for direct bacterial cell growth measurements. Despite impressive advances, most of these approaches start with culturing and enriching of the samples, followed by bacterial isolation and purification (thus, still slow). A recent advance in microfluidics introduced a mechanism to enable fast AST with clinical urine sample by flowing clinical samples along microfluidic channels and images cell elongation with high-resolution microscopy. However, trapping of the bacterial cells for accurate analysis requires prior knowledge of bacterial size, and the loading times are long for samples with low bacterial concentrations. Furthermore, this method is unreliable whenever antibiotics that influence cell elongation are present.

Developing a fast and accurate phenotypic ID/AST technology that can replace the current culture-based technologies faces several difficult challenges.

Challenge 1: The pathogen concentration in urine samples can be low (104-105 CFU/mL), so detecting individual bacterial cells in the sample without culturing and sample enrichment is challenging. High resolution imaging techniques (optical or AFM) can resolve single bacterial cells, but their imaging volumes and viewing areas are too small to visualize bacterial cells in such low concentration samples. For example, a 20× optical microscope has an imaging volume of 500×500×10 μm³, which contains ˜0.25 cells for a 105 CFU/mL sample. Sample preconcentration or microfluidic methods may overcome this difficulty, but it slows down the detection.

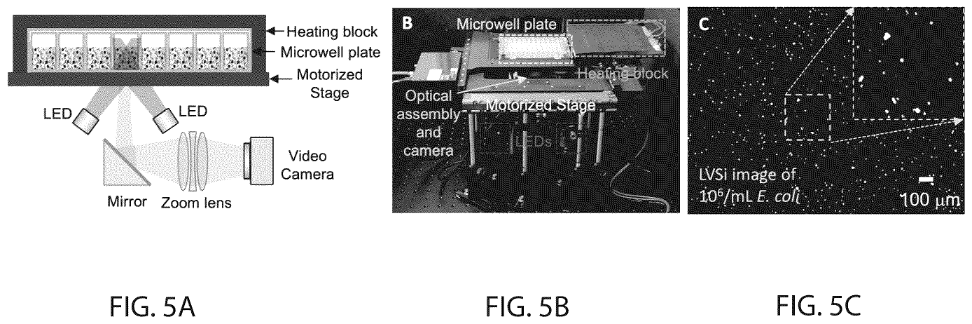

Accordingly, we have developed LVSi to image single bacterial cells at concentrations as low as 103 cells/mL without culturing or sample enrichment (FIG. 3). The technique creates a light slab to illuminate a large volume of the sample solution and image individual bacterial cells with a wide-view imaging system. The LVSi system used for our experiments has a viewing area of 1.8×1.5 mm² with imaging depth of 1 mm, corresponding to an imaging volume of ˜3 mm³, which contains ˜300 bacterial cells for a 10⁵ cells/ml sample. In addition to large image volume, LVSi images scattered light from the bacterial cells with low noise and low background, enabling nanometer precision tracking of the motion, morphology (size and shape), and growth of each cell. The shape and size of each cell are determined from the scattered intensity and its variation over time, similar to flow cytometry. Our results have shown that both side scattering and forward scattering imaging provide sufficient multi-phenotypic information of the bacterial cells for ID/AST. To provide enough throughput and lower the cost of consumables, we use a microplate-based back scattering imaging system in some implementations. Our data shows that the back scattering imaging not only provides comparable image quality but also enables a configuration that supports higher throughput detection using standard 96-well microplates (FIGS. 5A-5C).

Challenge 2: Culture independent ID and AST require working with raw urine samples that often contain various substances, including white/red blood cells, epithelial cells, casts, and crystals, and bacteria. Differentiating the pathogens from the other substances the samples based on images alone is challenging.

We have developed four strategies to address this challenge. 1) The collected urine sample is filtered to prevent substances larger than bacterial cells from entering the POCAST™ system. Using 5-μm filters removes substances larger than bacterial cells and effectively minimizes loss (<5%) of bacterial cells. 2) We track multiple phenotypic features of each cell, including motion, shape, size, and growth, in the absence and presence of antibiotics, and use these combined phenotypic features as an ID signature. 3) For UTIs, the top prevalent pathogens [uropathogenic E. coli (70%), Klebsiella pneumoniae, Staphylococcus saprophyticus, Enterococcus faecalis] have distinct phenotypic features (Table 1), and correctly identifying these four bacteriuria pathogens alone will cover 93% of all uncomplicated UTI and 84% of all complicated UTI cases. Furthermore, generating ID signatures for prevalent uropathogenic bacteria will enable rapid culling of clinical urine samples with non-clean-catch contaminating bacteria. 4) To further improve specificity, we include wells with selective growth media for the different bacteria and track associated bacterial growth rates (listed in Table 1). Finally, we note that antibiotics are prescribed according to groups of pathogens. In cases where precise ID is not possible, identification of possible pathogens is reported, allowing healthcare providers to make informed decisions and to promote antibiotic stewardship.

TABLE 1
Common UTI bacterial pathogens and their phenotypic features, selective media, and treatment drugs

| | Percent | | | | | Selective | | |
|---|---|---|---|---|---|---|---|---|
| | Un/ | | Gram | | | Media* for | First-line | Alternative |
| Species | Complicated | | Stain | Morphology | Motility | ID | treatment^{#} | treatment^{#} |
| Escherichia | 76% | 65% | − | Rod; single | Motile | MacConkey | Fluoroquinolone, | Aztreonam, |
| coli (UPEC) | | | | or pairs; 1.1- | | Broth¹ | 1-4^{th} Generation | β-lactam/β- |
| | | | | 1.5 μm × 2.0- | | | Cephalosporin, | lactamase |
| | | | | 6.0 μm | | | Trimethoprimd- | inhibitor |
| Klebsiella | 6% | 8% | − | Rod; 0.3-1.5 | Non- | | sulfamethoxazole | combinations, |
| pneumoniae | | | | μm × 0.5-5.0 | motile | | | Carbapenem |
| | | | | μm | | | | |
| Staphylococcus | 6% | 0% | + | Coccus, | Non- | m | 1-4^{th} Generation | β-lactam/β- |
| saprophyticus | | | | spherical; | motile | Staphylococcus | Cephalosporin, | lactamase |
| | | | | single, pairs, | | Broth | Trimethoprimd- | inhibitor |
| | | | | or clusters; | | | sulfamethoxazole, | combinations, |
| | | | | 0.5-1.5 μm | | | Fluoroquinolone | carbapenem |
| | | | | diameter | | | | |
| Enterococcus | 5% | 11% | + | Coccus, | Non- | SF Broth | Penicillin^{a}, | β-lactam/β- |
| faecalis | | | | spherical; | motile | | Amoxicillin^{a}, | lactamase |
| | | | | pairs or short | | | Ampicillin^{a} | inhibitor |
| | | | | chains, 0.6- | | | Vancomycin^{b} | combinations^{a}, |
| | | | | 2.0 μm | | | Daptomycin^{c}, | Carbapenem^{a}, |
| | | | | diameter | | | Nitrofurantoin^{c}, | Daptomycin^{b}, |
| | | | | | | | Fosfomycin^{c} | Nitrofurantoin^{b}, |
| | | | | | | | | Linezolid^{c} |
| Others | 7% | 16% | | | | | | |

*Available from Hardy Diagnostics, Remel, Becton Dickinson, ThermoFisher, and/or Sigma-Aldrich. Differentiated via motility characteristics and LVSi (E. coli - motile; K. pneumoniae - nonmotile). Since treatment is similar for Enterobacteriaceae, differentiation is not critical.
^{a}Penicillin- or ampicillin-susceptible Enterococcus sp;
^{b}Penicillin- or ampicillin-resistant Enterococcus sp;
^{c}Vancomycin-resistant Enterococcus (VRE);
^{#}Adapted from Mayo Clinic Antimicrobial Therapy Quick Guide, Mayo Clinic Scientific Press, 2008.

Challenge 3: Current phenotypic AST methods are primarily based on measuring the optical density, turbidity, or growth of all cells in the sample. While simple, these strategies are insensitive to phenotypic (morphological and physiological) changes of individual bacterial cells in the sample. Moreover, they require culturing and bacterial isolation, necessitating substantial time for bacterial growth prior to measuring the phenotypic change for AST. Collectively, these procedures add significant time and thus, compromise AST speed.

To address this challenge, we track individual bacterial cells with LVSi, such that phenotypic changes associated with antimicrobial activity against single cells are detected. This single-cell tracking capability provides sensitive detection of antibiotic-induced phenotypic changes for rapid AST. Single cell-based AST methods have been proposed, e.g., AFM and high-resolution optical microscopy, but their image volumes are small and require culture isolates or enriched samples. In contrast, our LVSi tracks low concentration bacterial cells in raw patient samples, which is a necessity for culture-independent AST.

Challenge 4: Antimicrobials induce multiple phenotypic changes of bacterial cells, including growth, metabolic-driven motion, and morphology. The gold standard broth microdilution assay and most of the emerging AST methods typically detect one phenotypic feature (e.g., growth) only. While growth is a valid phenotypic feature, AST based on growth alone can be slow for slowly growing bacteria. Incorporating additional phenotypic features, which can be more sensitive to antimicrobials, leads to faster and more reliable AST. For example, we have found that the metabolically-driven bacterial motion rapidly changes in response to antimicrobials, leading to faster AST than tracking growth alone. However, tracking multiple phenotypic features of each bacterial cell generates huge datasets, particularly when considering different bacterial strains and different antibiotics at multiple concentrations. Determining which combination of the multiple phenotypic features produce the fastest and most accurate ID/AST requires intelligent data analysis.

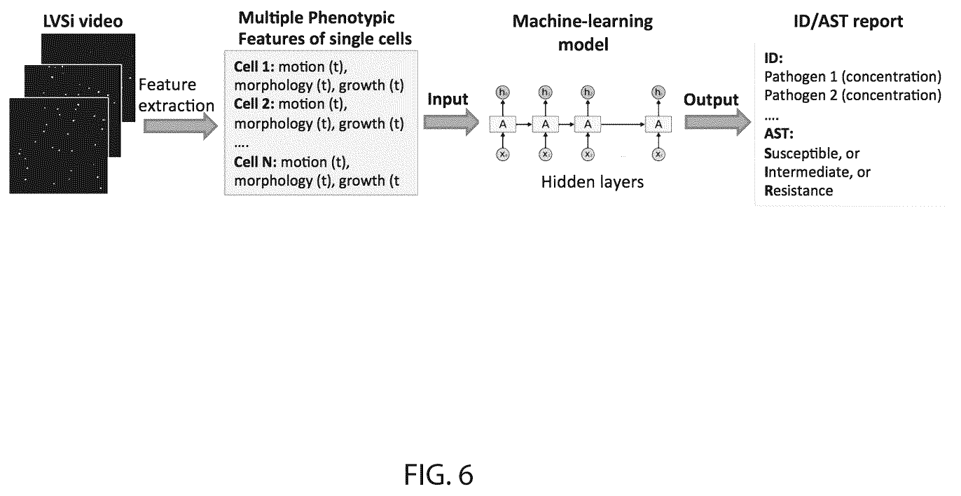

Accordingly, we use ML models for ID/AST by including all phenotypic features for every bacterial cell in the sample (FIG. 6). The model identifies each particle (bacterial cells or other substances) in the urine sample from the LVSi images and extracts all key phenotypic features for training. It is trained for ID and AST by comparing the outputs with gold standard reference results for both pure culture isolates and patient samples. The ML approach determines which phenotypic feature(s) provide the most accurate ID and AST, thus providing a universal platform for multiple pathogens and antibiotics at different concentrations. If the specific ID or accurate AST is not possible, the gold standard microbiology reference method will be used, and the results will be used to further train the ML model. This learning capability helps improve the accuracy of POCAST™ over time.

We have completed substantial studies to show that the POCAST™ technology can 1) detect viable bacteria and perform rapid AST (30-120 mins) for UTIs without culture isolation and sample enrichment; 2) determine and differentiate antimicrobial-susceptible and -resistant strains; and 3) detect bacteria and provide AST in raw patient urine samples. These foundational studies demonstrated the feasibility of POCAST™ for UTIs.

We develop a high-throughput POCAST™ system using standard 96-well microplate as consumables. We will test the key design parameters of high-throughput POCAST™ with a breadboard setup and spiked and clinical UTI urine samples. We develop a commercially viable high-throughput prototype POCAST™ instrument for rapid automatic pathogen ID/AST directly from urine samples for UTIs and validate the instrument using clinical urine samples.

### LVSi Technology

LVSi can Detect Bacteria without Culture-Based Bacterial Isolation and Sample Enrichment.

### Precision Tracking of Multiple Phenotypic Features of Single Bacterial Cells Using LVSi.

To detect bacteria directly in a patient urine sample without pathogen culturing or isolation, we track the growth, morphology, and motion of each single particle (bacterial cell or another substance) by measuring the number, position, and intensity from the LVSi video over time. These features provide key “signatures” to identify the pathogen. We have developed algorithms to precisely extract these features from the LVSi videos:

### Metabolically-Driven Motion Tracking.

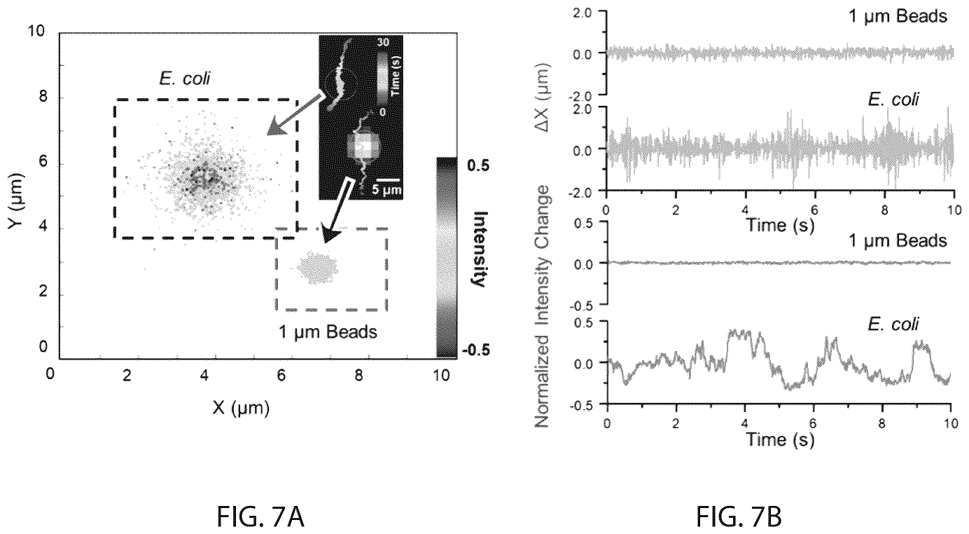

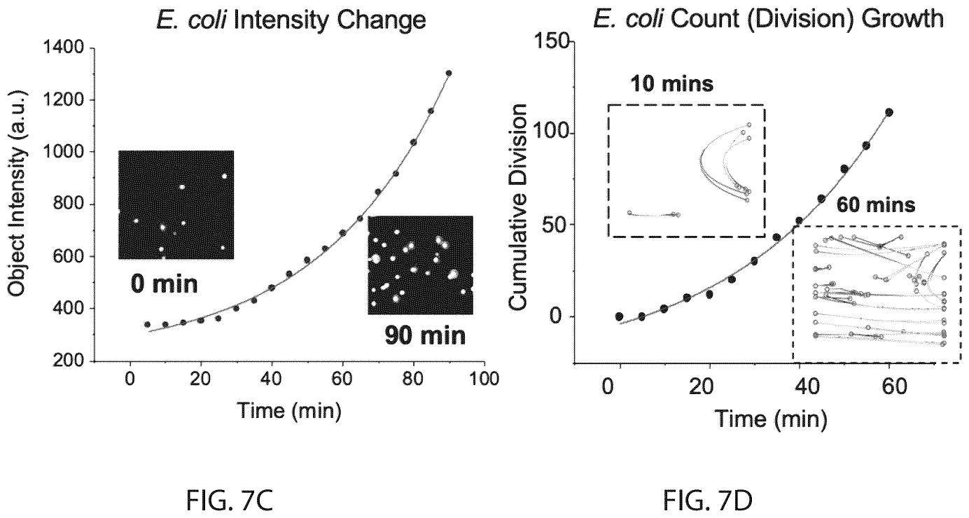

We have achieved ˜10 nm tracking precision in dilute urine samples (10⁴-10⁵ cells/mL) (FIG. 7A). This precision can resolve the Brownian motion of a single particle, thus allowing us to identify bacterial cells based on their motility and other metabolically-driven motions (e.g., swimming, tumbling). FIG. 7B top plot shows motion tracks of bacterial cells (E. coli) and polystyrene particles (as references) to differentiate between the metabolic and Brownian motions.

### Morphology Tracking.

The particle image intensity and its variation over time provides detailed information about the size and shape of the particle based on Mie scattering theory (FIG. 7B bottom plot). It also provides information on the growth and metabolic activities (e.g., movement, motility, tumbling, rotation) of a bacterial cell. The peak intensity provides the length of the bacterial cell, and the valley intensity reflects width. The frequency of the intensity variation is related to the motility of the bacterial cell, which helps differentiate a bacterial cell from non-bacterial object or a dead cell.

### Growth (Division) Tracking.

Another important feature precisely extracted by LVSi is growth as demonstrated by changes in cell size and division. Increases in cell size leads to increased intensity (FIG. 7C), while cell division results in the splitting of one spot into two and increases in the total number of spots (FIG. 7D). By tracking growth rate in selective culture media, we can further expand pathogen identification accuracy.

### Detect UTI Pathogens Using Machine-Learning Classifier.

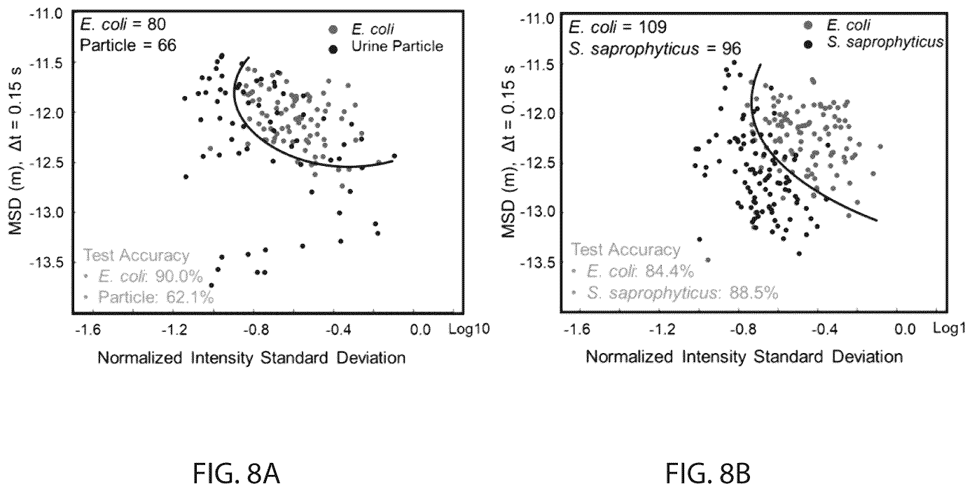

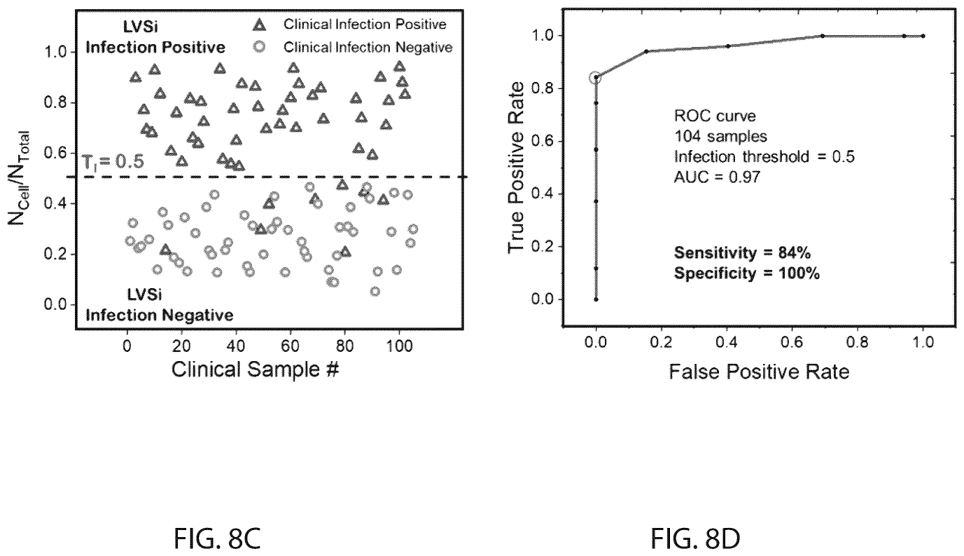

We have developed ML-based classifiers that combine all features for each of the particles in the sample, and successfully achieved rapid and accurate detection of E. coli (UPEC) from non-bacterial particles in urine samples (FIG. 8A), and bacterial pathogen differentiation (FIG. 8B). Using this method, we detected positive UTI infections from 104 clinical urine samples in 10 minutes with 84% sensitivity and 100% specificity. (FIG. 8C, D).

Fast AST without Culture Isolation and Sample Enrichment

Using LVSi, we have measured pure bacterial cultures, urine samples spiked with E. coli, and patient urine samples with and without UTI infections (from Mayo Clinic) using antibiotic-resistant and -susceptible bacteria and multiple antimicrobial drugs. By precisely tracking and counting phenotypic changes at single cell level with different doses of antibiotics, we are able to determine the minimum inhibitory concentration (MIC) of antimicrobials directly from urine samples and achieve fast AST (<120 mins). Key results are highlighted below:

### Metabolically-Driven Motion Changes.

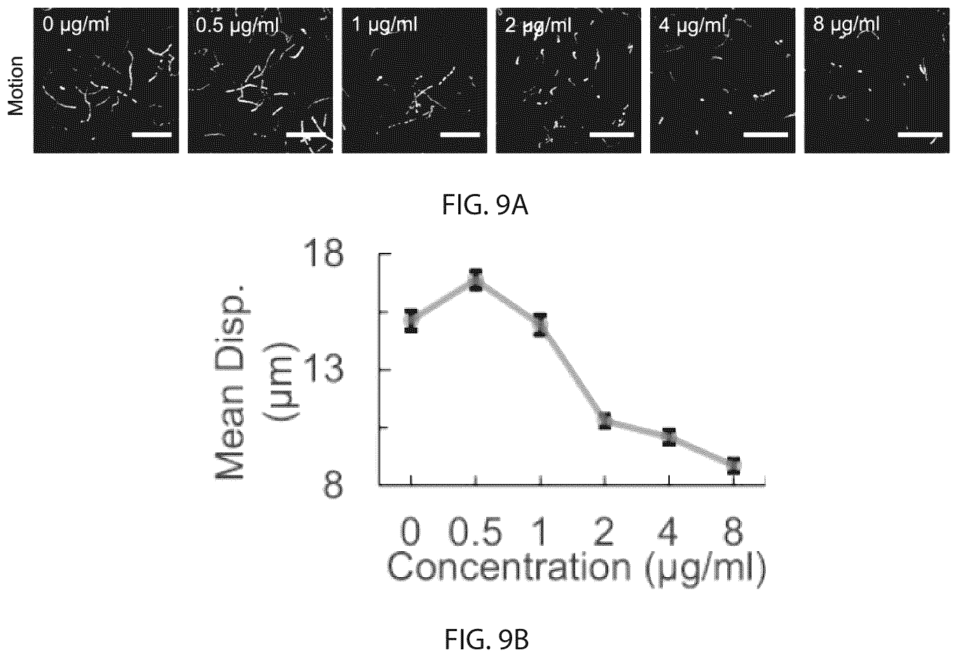

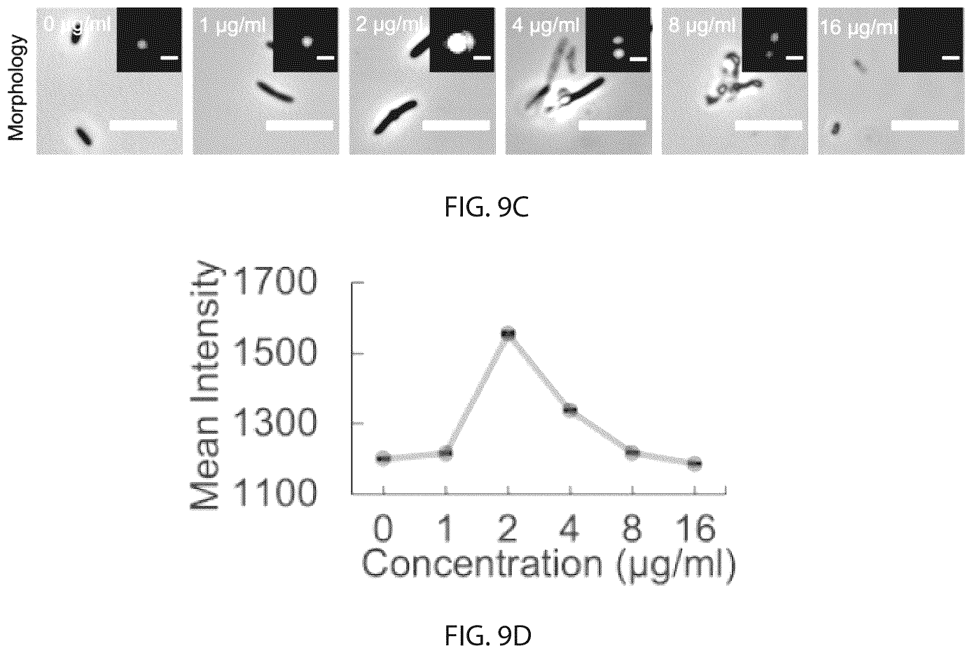

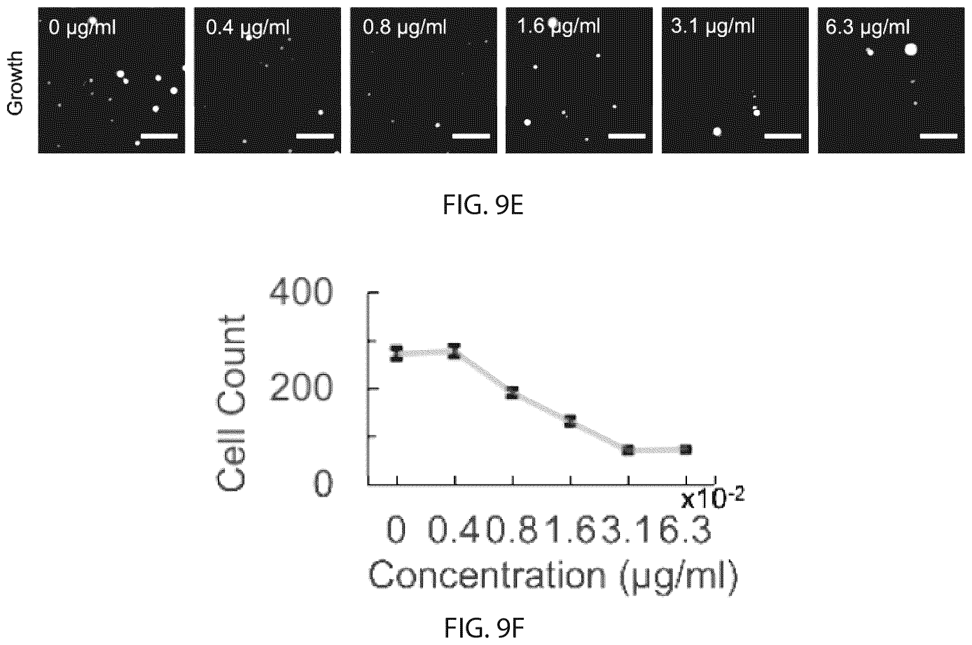

Antimicrobials induce bacterial motion changes by affecting swimming, tumbling, twitching, swarming, gliding, spreading, and physiological responsiveness. Although some UTI pathogens are non-motile, Staphylococcus aureus spreads using dendrites that mimic gliding motility and K. pneumoniae exhibits fimbriae-mediated motions. For example, polymyxin B (PMB), a polypeptide antimicrobial that disrupts the cell membrane, leads to decreased swimming and tumbling (FIG. 9). We tracked motion changes by measuring the displacement of each bacterial cell with nanometer scale resolution and obtained antimicrobial dose curves within 30 min (FIG. 9B). From the dose curves, we recorded an MIC value of 1-2 μg/mL, which corresponds to the MIC obtained using the gold standard broth dilution method.

### Morphology Changes.

Antimicrobials induce morphology changes in cells, including size and shape (bulge formation, elongation, shrinking due to cell lysis, etc.). Some antimicrobials (Table 2) lyse cells, inducing cell shrinkage, while others inhibit cell division, causing continuous filamentation and size increases until cell lysis and death. Therefore, by tracking morphological changes, we can also determine the antimicrobial effect for AST. FIGS. 9C-D show E. coli morphological changes upon exposure to different concentrations of ampicillin. With 2 μg/mL ampicillin (determined MIC), the bacterial cells elongate (FIG. 9C), as expected and as shown by an increase in the mean intensity of each bacterial cell (FIG. 9D). At higher doses (>4 μg/mL) of ampicillin, most cells are lysed, which is detected from the decreased mean intensity (FIG. 9D, dim spots in the LVSi images).

### Growth (Division) Rate Changes.

Bactericidal and bacteriostatic antimicrobials inhibit the growth (division) of bacteria. Our LVSi resolves individual bacterial cells, enabling tracking of division of individual cells within the sample. We tracked division and growth of E. coli upon exposure to ciprofloxacin (FIG. 9E) and observed that the growth rate and cell counts decrease with increasing doses (FIGS. 9E-F).

Collectively, these results showcase strong evidence that the MIC concentrations of different antimicrobial classes (PMB, ampicillin, ciprofloxacin) can be determined using different phenotypic responses for three different E. coli strains (to mimic potential strain diversity in clinical samples).

TABLE 2
FDA Recommended antimicrobials for UTIs, their
drug mechanisms and phenotypic changes

| | | | | Phenotypic |
|---|---|---|---|---|
| Mechanism | Class | Antimicrobials^{#} | | Changes |
| Cell wall | β-lactam | Amoxicillin- | Cefazolin^{a, 2} (1^{st} GC) | Morphology, |
| synthesis | | clavulanate^{b, 5} | Cefepime^{b, 2} (4^{th} | Growth, |
| | | Ampicillin^{a, i, 1} | GC) | Motion |
| | | Aztreonam^{c, 4} | Cefotetan^{b, 2} (2^{nd} | |
| | | Doripenem^{b, 3} | GC) | |
| | | | Ceftriaxone^{b, 2} (3^{rd} | |
| | | | GC) | |
| | | | Cefuroxime^{b, 2} (2^{nd} | |
| | | | GC) | |
| | Glycopeptide | Vacomycin^{f, j} | | |
| | | Fosfomycin^{d, l} | | |
| DNA/RNA | Fluoroquinolone | Ciprofloxacin^{b, g, l} | Norfloxacin^{d, h, l} | Morphology, |
| synthesis | Rifamycin | Rifampin^{f} | | Growth |
| Protein | Aminoglycoside | Amikacin^{b} | Tobramycin^{a} | Growth |
| synthesis | | Gentamicin^{a, g, k} | | |
| | Tetracycline | Tetracycline^{c, f, l} | | |
| | Macrolide | Erythromycin | | |
| | Oxazolidinone | Linezolid^{f, j} | | |
| Folate | Sulfonamide | Sulfisoxazole^{d, h} | Sulfamethoxazole^{b, e} | Growth |
| synthesis | Diaminopyrimidine | Trimethoprim^{b, d, e, h} | | |
| Permeability | Lipopeptide | Daptomycin^{f, j} | | Morphology, |
| | | | | Growth, |
| | | | | Motion |
| Multiple | | Nitrofurantoin^{d, h, l} | | Morphology, |
| | | | | Growth |

*Adapted from Antimicrobial Chemotherapy 7^{th} edition, Oxford University Press, 2015;
^{#}Grouped according to CLSI M100-S26, 2016; GC, generation cephalosphorin;
^{a}Group A, Enterobacteriaceae;
^{b}Group B, Enterobacteriaceae;
^{c}Group C, Enterobacteriaceae;
^{d}Group U, Enterobacteriaceae;
^{e}Group A, Staphylococcus spp.;
^{f}Group B, Staphylococcus spp.;
^{g}Group C, Staphylococcus spp.;
^{h}Group U, Staphylococcus spp.;
ⁱGroup A, Enterococcus spp.;
^{j}Group B, Enterococcus spp.;
^{k}Group C, Enterococcus spp.;
^{l}Group U, Enterococcus spp.; (Group A antibiotics: Primary test and report; Group B antibiotics: Optional primary test and report; Group C antibiotics: Supplemental test and report; Group U antibiotics: Supplemental for urine only)
¹Penicillins;
²Carbapenems;
³Cephalosporins;
⁴Monobactams;
⁵β-lactamase inhibitor combinations.

Susceptibility Profiling Using Urine Samples Collected from UTI Patients

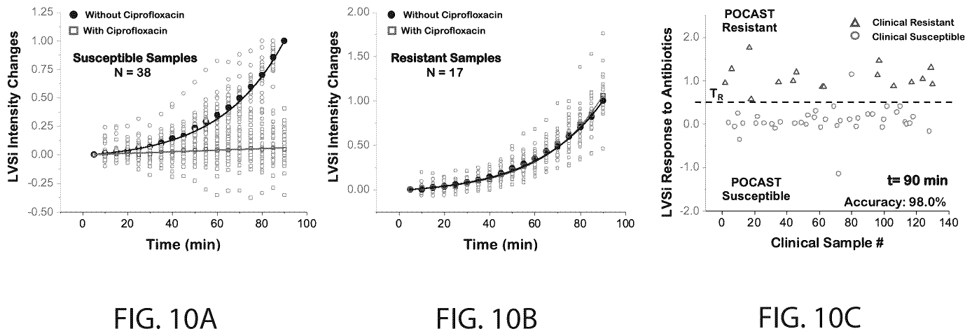

To demonstrate rapid AST directly from urine samples, we measured UTI infection positive clinical urine samples collected from UTI patients at Mayo Clinic. Samples were passed through 5 μm syringe filters and mixed with culture medium only (control) or with medium containing antibiotics at the clinical breakpoint concentration (CLSI standard), then immediately analyzed with a dual channel LVSi setup (with no other sample pretreatment) for 90 minutes. One channel measures the control sample, and the other channel measures the sample mixed with antibiotics. By analyzing phenotypic changes of bacteria in the samples, represented by count, 14 division events, or intensity changes in the LVSi videos, we quantified the growth status of the bacteria and the effectiveness of the antibiotics. As an example, FIG. 10 shows the LVSi results of 55 UTI positive urine samples by tracking the total particle intensity changes in response to ciprofloxacin, where susceptible samples show clearly inhibited growth (no intensity increase) compared to the controls (FIG. 10A), and resistant samples show similar growth rate as controls (FIG. 10B). The 90-minute LVSi AST results are in 98% agreement with both BD Phoenix results from 2-3 day (culture isolation, enrichment, and AST) clinical microbiology lab testing and the overnight on-site parallel AST plating validation.

### Data on Microplate Based High Throughput LVSi Technology

The LVSi technology results presented above were obtained using disposable off-the-shelf microcuvette sample holders via side or forward scattering imaging. While this approach is convenient for proof-of-concept studies, it is difficult to increase throughput and enable simultaneous testing of a whole panel (˜8) of UTI antibiotics (Table 1), where each drug needs to be tested with multiple doses. Therefore, to provide sufficient throughput for testing all common UTI antibiotics in a single run and minimize consumable costs, we develop a microplate-based back scattering imaging system, using a standard flat bottom 96-well polystyrene microplate. In principle, back scattering imaging provides similar object information as side or forward scattering imaging. However, back scattering imaging has the additional advantages of increased throughput, avoiding the image distortion caused by the curvature at the air/liquid interface, and leaving the top of the plate available for efficient temperature control.

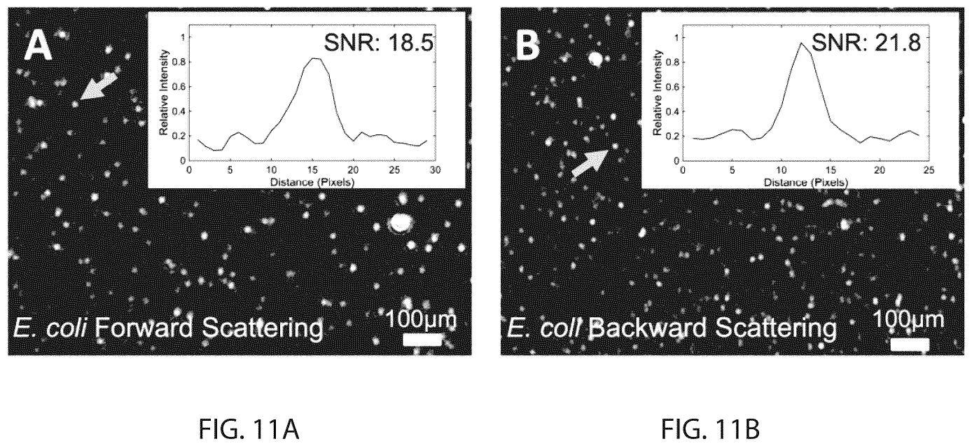

We conducted tests of back scattering imaging of E. coli samples in a 96-well microplate. We used two 660 nm LED lights as illumination sources and collected the back scattering images with a zoom lens set at 3× and CMOS camera, as shown in FIG. 5. The microplate is placed on a motorized microscope stage, and the temperature of the microplate was controlled at 35±2° C. (CLSI standard). Using this testing setup, we obtained back scattering images of E. coli in the microplate wells with similar contrast as the cuvette-based forward or side scattering LVSi systems (FIG. 11).

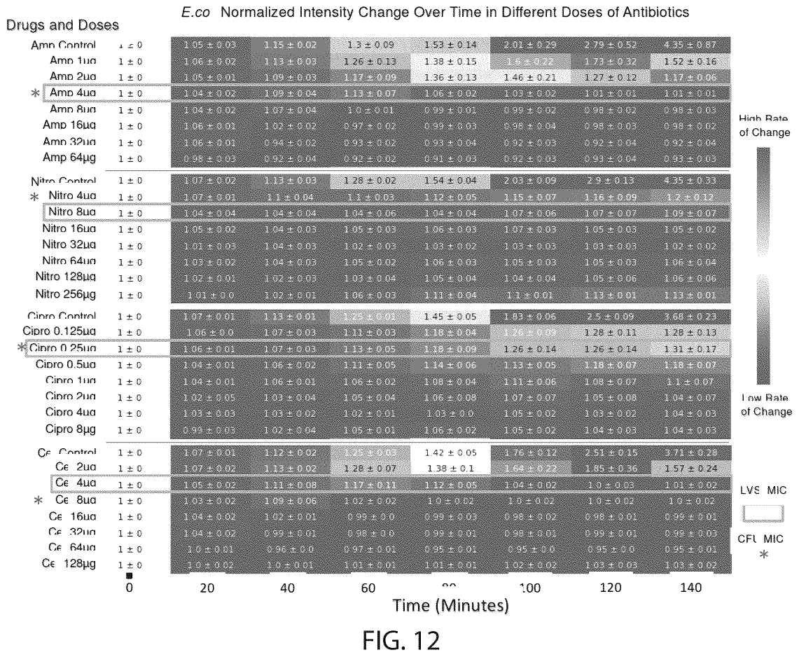

To demonstrate the high-throughput capability of microplate-based LVSi technology, we performed AST measurements of E. coli with 4 different antibiotics, each at 8 different doses with 3 replicates. The normalized mean particle intensity changes of each dose are shown in FIG. 12. As shown in FIG. 12, the LVSi-determined MIC concentrations, defined when bacterial growth stops and the corresponding LVSi intensity remains static, are equal to or within one dilution factor of the CFU MIC values. These results show that the microplate-based back scattering LVSi technology can provide accurate, high-throughput AST measurements.

### Phase I Research Plan: Develop Microplate-Based High-Throughput LVSi Technology for POCAST™

The data described above demonstrates the feasibility of the microplate POCAST™ for ID/AST of common UTI bacteria within 3 hours using urine samples directly without culturing or sample enrichment. We build and test a microplate-based breadboard back scattering LVSi setup to determine key design parameters for rapid UTI POCAST™ ID/AST.

### Develop a Microplate-Based Breadboard Back Scattering LVSi Setup

We develop a 96-well microplate-based setup for multiplexed tracking of bacterial cells at UTI-relevant concentrations in urine samples, which enables rapid ID/AST of common UTI pathogens with a panel of UTI antibiotics in a single test.

### Optical and Mechanical Design.

As illustrated in FIG. 5A, the microplate is precisely positioned on a linear motor X/Y translation stage [e.g., a Zaber X-ADR-AE Series XY microscope stage (Zaber, Vancouver, CA)]. The stage is programmed to move through all wells so that short (5-10 s) LVSi videos are captured for each well sequentially. To detect enough bacterial cells at clinically relevant UTI concentrations in raw urine samples, the LVSi system with need an image volume of ˜10 mm³. The image volume is determined by the viewing size and focal depth of the optics. Two parallel LED light sources (e.g. M660L4 from Thorlabs, Newton, NJ) are positioned at the bottom of the microplate along the edges of the well at a small angle to provide uniform illumination covering a single well, while avoiding the collection of the reflected light from bottom of the well. The backscattered light from the bacteria in the well is captured using a variable zoom lens (e.g. Navitar 12× zoom lens from Thorlabs), coupled with a USB3 CMOS camera (e.g., Blackfly S BFS-U3-04S2M, FLIR, Wilsonville, OR). The variable zoom capability can reduce the image volume for higher concentration samples. The numerical aperture of the zoom lens at 2× zoom is ˜0.03, corresponding to spatial resolution of 10-15 μm. Though this resolution is insufficient to resolve single bacterial cell morphology directly, LVSi cleverly determines the morphology of a cell from the intensity of the image (appears as a bright spot).

### Temperature Control.

For proper growth of the bacterial cells, the temperature must be tightly controlled at 35±2° C. Backscattering imaging permits direct placement of resistive heating pads and thermal probes on top of the 96-well plate enclosure for accurate temperature control and feedback in real time. The thermal enclosure covers both the top and sides of the plate, ensuring stable temperature conditions are maintained. Temperatures in each well are measured with a thermal imaging camera and/or single well measurement with a thermometer in a calibration step to tune the set temperature of the heating pad. The inside of the resistive heating pad casing is painted with a specialized low reflection flat-black film to minimize scattering/reflection of light for clean collection of scattering images. The whole setup is placed on an aluminum optical bread board and enclosed by thermal-isolating foamboard for effective temperature control.

### Determine Key Design Parameter for Track Single Bacterial Cells in Urine Samples.

Key parameters to be tested and determined include optimal illumination light configurations that provide uniform illumination and minimal reflection. Imaging focal plane positions, exposure times, frame rates, and short video lengths will be assessed for optimal particle tracking and rapid detection, along with the stability and uniformity of microplate temperature control. To test the performance of the microplate-based LVSi system, we will spike selected UTI pathogens, such as UPEC CFT073, ciprofloxacin-resistant CFT073, or ampicillin-resistant CFT073, into normal urine (from BioreclamationIVT) at 10⁴-10⁸ CFU/mL concentrations. Thereafter, the spiked urine will be mixed with cation-adjusted Mueller-Hinton Broth (CAMHB) medium (CLSI standard) in 1:1 ratio and cells will be analyzed with LVSi. We also test ESBL K. pneumoniae ATCC 700603, ESBL E. coli ATCC 51446, S. saprophyticus ATCC 15305, and vancomycin-resistant E. faecalis ATCC 51299 in the presence and absence of antibiotics to assess phenotypic changes and responsiveness of diverse uropathogens to different antibiotics, test the performance of the optics, and calibrate the LVSi video. After tuning the optics, we test with patient samples from Mayo Clinic. We study different phenotypic features from the LVSi video. Growth (multiplication/cell division) is measured as an increase in the number of spots (cells). Cell morphology and size changes are determined from the intensity and its characteristic variation over time, and cell motion is tracked with nanometer precision by quantifying the position with a Gaussian fitting algorithm (FIG. 7A).

### Potential Pitfalls and Alternative Approaches.

For common all-clear flat bottom microplates (e.g. Corning #3370, ˜$4 each), illumination light may reflect and scatter from nearby wells and increase the background of the LVSi image. Furthermore, light reflection and scattering interference could differ for interior wells and wells located along the edge of the microplate. Our testing demonstrates that we can indeed obtain accurate LVSi back scattering imaging of E. coli in clear microplates (FIG. 11). However, if image contrast is insufficient for smaller bacteria (e.g., S. saprophyticus), then we can switch to black microplates with flat clear bottoms to eliminate the background light from nearby wells, albeit with slightly higher consumable costs (e.g., Corning #3603, ˜$10 each).

### Develop Image Recording and Processing Algorithms for the Microplate-Based LVSi

### Develop Automated Image Recording and Feature Extraction Algorithms for Recording of Whole Plate LVSi Short Videos at Multiple Time Points and Extract Key Phenotypic Features.

We develop imaging recording algorithms synchronized with the microplate translation, for automated recording of short videos of each well sequentially, at predefined intervals, so that each well can obtain multiple short videos at different time points using a single camera. The algorithms are implemented with Python and utilize Zaber Motion Library for translating the wells with a Zaber microscope stage. We also extract phenotypic features from the LVSi videos. The LVSi video contains growth, morphology, and motion changes of individual bacterial cells, but feeding the entire video into the ML algorithm is computationally expensive and impractical. Moreover, the number of samples needed to train such a model would be prohibitive. To significantly minimize these burdens, we only extract specific features: intensity (I(t,i)), and position (x(t,i) and y(t,i)) of each imaged object, where t denotes time and i, the i^{th} imaged object (bacterial cell or an impurity). Intensity I(t,i) provides rich information about the size, shape, and growth (cellular changes). Positions, x(t,i) and y(t,i), describe the motion associated with motility and other metabolic-driven motions.

### Train the ML Model for Fast ID and AST.

After obtaining accurate feature representation, we develop an effective ML model for ID and AST. We have evaluated SVM, Convolutional Neural Networks (CNN), and Long-Short Term Memory (LSTM) ML algorithms in our studies with selected UTI pathogens and drugs. These models generally performed well with each model displaying unique advantages. For example, LSTM is particularly effective for processing time-dependent phenotypic features and accurately identifies different shaped bacteria. We advance these ML models for ID/AST of common UTI pathogens and antibiotics using the high-throughput data generated in this project. Quality and quantity of the data available for training the models are critical benchmarks for technological advancement. We have accumulated substantial forward and side scattering LVSi data of UTI patient urine samples with four common UTI antibiotics (ampicillin, nitrofurantoin, ciprofloxacin, and cefazolin). We obtain additional back scattering LVSi data with the high-throughput microplate LVSi system using clean urine spiked with bacteria and urine samples collected from patients with and without UTIs, where the ground truth for each sample is obtained using gold standard culture-based reference technologies. We train and test these back scattering data with selected ML models and compare the model performance with results from the forward and side scattering image data. For ID, the output of the ML model includes bacterial pathogens at varying concentrations. For AST, the training data includes different antibiotics for UTIs (Table 2), and the output includes Susceptible, Intermediate, and Resistant. The training dataset is fed into the ML model and processed over multiple iterations. After training the ML model, we test it using clean urine spiked with bacteria and urine samples collected from patients with and without UTIs.

### Potential Pitfalls and Alternative Approaches.

The microplate-based system has a shortened total imaging time (5-10 s) for each well, due to the need to imaging all 96 wells in sequential mode for multiple time points during the 1-3 hr assay time. Our data shows that the bacterial LVSi intensity and count changes in response to four common UTI antibiotics can predict the MIC values with 9 s videos at 10 fps (FIG. 12). We test different video lengths, frame rates, and measurement intervals to determine optimal settings. And if needed, additional cameras could be added to the system for parallel imaging, so that the total measurement time for the whole plate can be reduced to avoid time bias of different wells while individual wells can be imaged with sufficient length for phenotypic feature tracking. Optionally, miniaturized one or more miniaturized cameras and/or light sources can be used. We also record and calibrate the time delay between the wells in the image processing algorithms.

Test the Plate-Based LVSi System for ID/AST for UTIs with a Pilot Study Using Clinical Urine Samples

Epidemiology evidence shows that the most common pathogens in UTIs are uropathogenic E. coli (70%), followed by K. pneumoniae, S. saprophyticus, and E. faecalis. Correctly identifying these bacteria will account for most UTI cases (Table 1). Each bacterium has one or multiple distinct phenotypic features, including but not limited to Gram-positive or Gram-negative morphology, flagellate- or non-flagellate-mediated motion, shape, and size. LVSi provides low-noise tracking of these distinctive phenotypic features of single bacterial cells with high precision. For example, rod-shaped, uropathogenic E. coli produces periodic intensity fluctuation due to flagellar rotation, while spherical S. saprophyticus has an entirely different intensity vs. time profile. The Gram-negative bacilli UTI pathogens are rods, while the Gram-positive cocci pathogens are spherical. By detecting the intensity fluctuation, we can differentiate between Gram-negative and Gram-positive UTI pathogens. By analyzing the intensity profiles, we extract quantitative information, such as rotational frequency of the bacterial cells, viscous damping, and metabolic-related motion, which are related to bacterial size and length. When a bacterial cell grows, the size change leads to an increase in the image intensity. The detailed phenotypic features extracted from LVSi carry rich information for precise identification of the common UTI pathogens. Our LVSi results showed >85% identification accuracy and 98% infection detection (FIG. 8), and <2 hours AST (FIG. 10) with UTI patient urine samples. We improve the performance with the ML model for automatically extracting these phenotypic features for each cell and determine the best combination of features for fast and accurate ID and AST. In addition, we test different selective growth media (Table 1) and track the growth in the media to further improve the specificity of ID and AST.

We also design and build up to three (3) prototype POCAST™ instruments following the design criteria determined above. Two units are for preclinical testing at ASU and Mayo Clinic, and one unit will be for off-site continued development, software refinement, and service as a backup unit. Developing POCAST™ into a commercial product involves multiple iterative cycles of development and testing.

### Design and Build a Commercial Prototype POCAST™ Instrument for Preclinical Testing

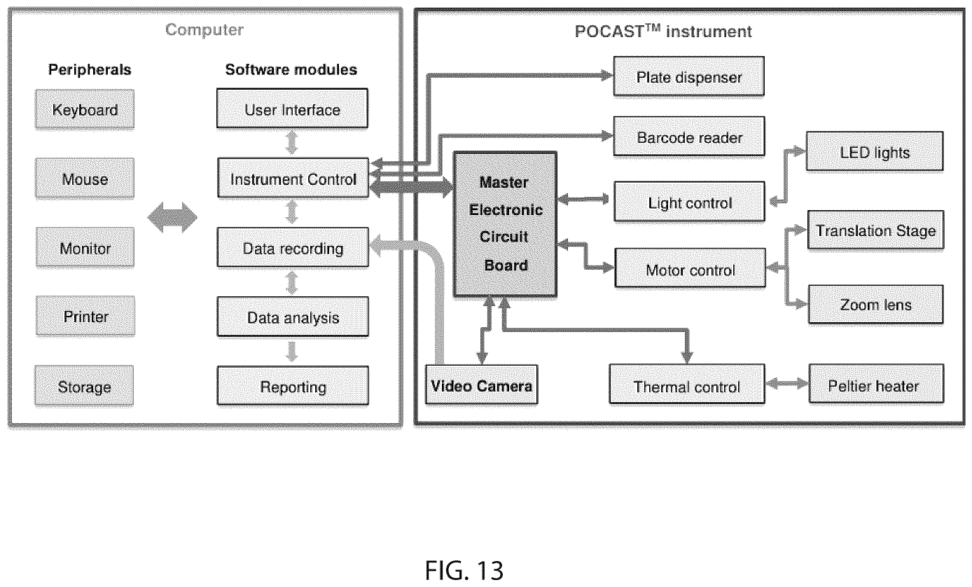

We design and build commercially viable prototypes following the design criteria determined above. The system includes microplate sample dispenser, barcode reader, detection optics, temperature and plate motion control hardware, system control, user-interface, and data analysis software (FIG. 13).

### Microplate Dispenser.

The system includes a microplate dispenser for automated delivery of urine samples to the 96-well sample plate, as shown in FIG. 4. The dispenser uses disposable tips or contact free dispensing to avoid cross contamination between different samples and includes auto shaking function for mixing the samples. We can use an off-the-shelf dispenser for adaptation and integration, such as FlexDrop Plus non-contact dispenser or VIAFLO multichannel Electronic Pipettes plus assist. The sample filled microplate is loaded to the POCAST instrument, with a mechanism to load and secure the plate in the instrument with proper positioning for ID and AST.

### Optics and Detection Components.

Two high power LEDs with collimation lens are used to illuminate a well of the sample plate from the bottom for back scattering LVSi. The optical assembly includes a motorized tunable zoom lens to adjust the image volume over two orders of magnitude to cover samples with concentrations of 104-10⁸ CFU/mL. A fast USB3 CMOS imager is used to collect back scattered light from the bottom of each well. We have tested a USB3 CMOS camera (FLIR Grasshopper GS3-U3-23S6M-C) with up to 163 fps capture rate at 1920×1200 pixels resolution for simultaneously tracking of up to 10,000 individual bacterial cells. The USB3 connection provides open communication and offers sufficient bandwidth for the data streams. The microplate is mounted on a motorized x-y translation stage so that all wells can be imaged sequentially.

### Master Control Electronics.

A main electronic board for centralized connectivity and communications of the system is designed and manufactured. It consolidates all connectors with internal and external devices, such as motor controllers, temperature controllers, data acquisition board, and camera. In this way, only a few cables will simply connect the system to the PC. A 6000-series data acquisition card from National Instruments Inc. is used to control digital and analog I/O lines and provide a high-resolution clock for syncing data sources and signals. The PC is a desktop workstation with sufficient speed and storage to meet the data processing need, e.g. Intel i9 processor, 64 GB memory, and a 4 TB solid state disk.

### Actuator Control.

The system is equipped with motorized stages for microplate positioning, illumination and imager positioning, focus, and zoom. Precise actuation of these components is important for rapid and accurate ID/AST measurement. A suite of actuators with integrated controllers from Oriental Motors will be implemented.

### Temperature Control.

CLSI standards require pathogen ID and AST at 35±2° C. The system supports a sample temperature control range from 20-40° C. with stability of +0.2° C., thus enabling future testing of bacterial strains that require lower incubation temperatures. Precision Peltier heating/cooling elements are mounted to a top aluminum casing to enclose the microplate. The instrument is also well insulated and covered to reduce thermal influences by convective heat flow from the ambient environment. The PTC series thermal controllers from Wavelength Electronics are used to control temperatures of various elements.

### Develop System Control and Data Analysis Software for the POCAST™ Prototype

We develop software, including the following modules: user interface, instrument control, data collection, ML-based data analysis, and ID/AST reporting with C++ and Python (FIG. 13).

The interface module provides a user-friendly interface with step-by-step wizards guiding the user through the entire measurement workflow, including sample preparation, patient information registration, parameter selection, choice for real time display of LVSi images, intermediate ID/AST results, and final ID/AST report generation. The instrument control module sends operational commands to the instrument for plate dispensing, temperature control, and translating the detection system through the wells of the cartridge for sequential measurement at set time intervals. The data collection module provides real time transfer of all LVSi video data from the instrument to the computer along with other necessary information, including cartridge ID, temperature, etc. The data analysis module incorporates the developed ML algorithms, to analyze the LVSi video for pathogen identification, extract phenotypic features, and analyze antimicrobial susceptibility. We continue the training and optimization of the ML models with additional data. The reporting module provides a comprehensive report of the sample, including identified pathogen name and initial concentration, as well as susceptibility/resistance status to the drug panel loaded in the cartridge in the form of CLSI interpretive standards: Susceptible, Intermediate, and Resistant. Confidence level of all results is provided, along with recommended dosage of drugs and/or additional tests. Data management: We also implement an interface with electronic health records (EHR) system following the Health Level-7 (HL7) standards for automatically uploading POCAST™ reports to EHR databases. In the final product, raw LVSi videos are deleted after generating the report. We can also keep all raw videos for research and validation purposes, as well as for preparing FDA applications. Representative images for each sample are retained for user review and assessment and for troubleshooting instruments in routine use. We keep all data organized in an encrypted network attached storage system.

Develop POCAST™ Microtiter-Plate with Preloaded Antibiotics and Media

We design, fabricate and test POCAST™ microtiter-plate with preloaded selective media and common UTI antibiotics as consumables for the POCAST™ instrument. The design allows for ID/AST of UTI pathogens from urine samples in a single rapid test. The pre-dosed and lyophilized format ensures convenience storage of the fabricated microplate with extended shelf life, eliminates the need for complex preparation steps, and minimizes test variability.

### Preparation of Antimicrobial Drug Solutions and Coating Plate.

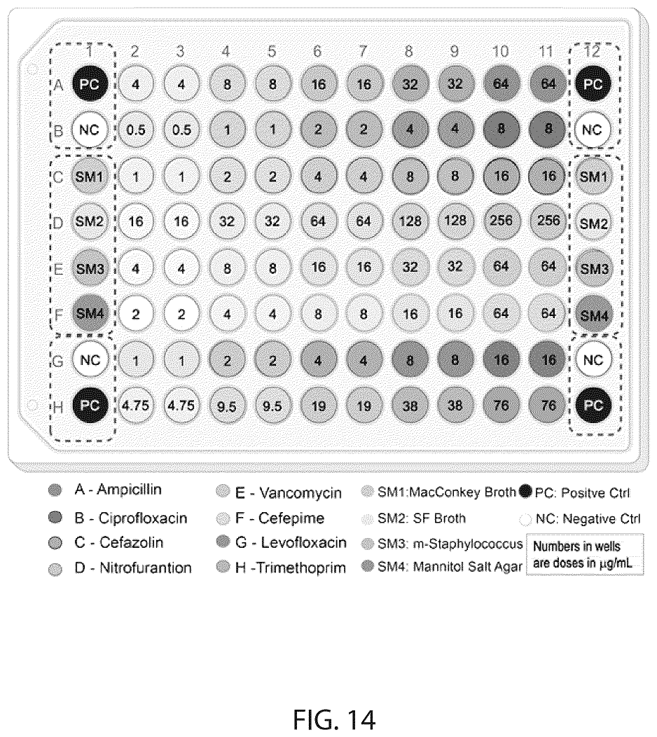

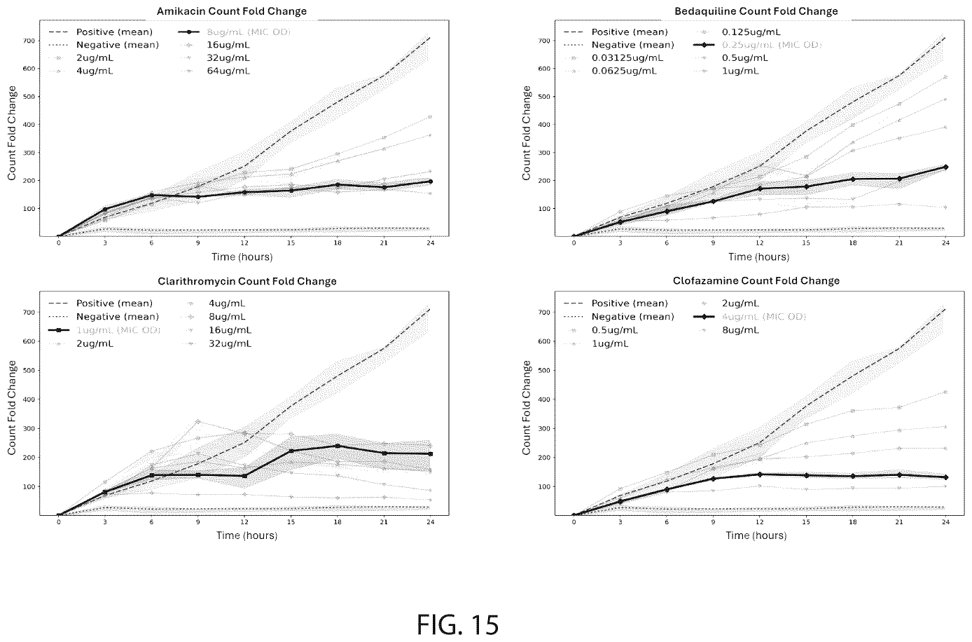

FIG. 14 shows a layout of the microplate, which includes 8 common UTI antibiotics in 5 doses with 2 experimental replicates, covering all CLSI-recommended Susceptible, Intermediate, and Resistant categories. The plate also includes positive controls without any antibiotics, negative controls without sample, and selective media (SM1-4) for accurate ID of the four most common bacterial pathogens in UTI patient samples (Table 1). The preparation of antibiotic solutions must incorporate the solubility of each drug. Hydrophobic drugs, such as ampicillin and cefazolin, are dissolved in water to create stock solutions. For hydrophilic drugs, like ciprofloxacin and nitrofurantoin, an initial dissolution in dimethyl sulfoxide (DMSO) is used before further dilution in water to achieve the desired concentrations for testing. This approach ensures that each drug is properly solubilized and can be accurately dispensed into microtiter plates for susceptibility testing. In some instances, for example, ciprofloxacin is dissolved in 0.1M HCl. Antibiotic stock solutions are sterilized by passing through a 0.22 μm filter. Each antibiotic is diluted to preset doses with sterile CAMHB medium and dispensed 200 μL into the corresponding well of sterilized 96 well microplates as shown in FIG. 14. The control wells are dispensed with 200 μL CAMHB only, and the selective media wells are dispensed with 200 μL of corresponding selective media as shown in FIG. 14. The dispensing will be operated in a sterile environment. The filled plate will be lyophilized in a freeze dryer to preserve the drugs and medium for long-term stability. The lyophilized plates are vacuum sealed with light shielding sterile sealing films to prevent moisture absorption and contamination.

### Testing of Storage Conditions.

To find the optimal storage condition and shelf life of the POCAST™ microtiter plate, the sealed plates are stored under different conditions, at 25° C., 4° C., −20° C., and −80° C. The stability of the loaded microplate is evaluated at various time points (0.5, 1, 3, 6, 9, and 12 months) to determine shelf life. At each time point, four plates at each storage condition are measured. Normal urine spiked with one of the four common UTI pathogens listed in Table 1 at 2×10⁵ CFU/mL is filtered and placed in 20 mL disposable vials. Each well of the plate is inoculated with 200 μL of the sample (water for negative control wells) via the auto dispenser. Plates undergo a standard shaking process to ensure the complete rehydration of the antibiotics and medium. The plates are then tested in the POCAST™ instrument. The results are compared to freshly prepared control plates to detect any reduction in media and drug activity. The storage shelf life of the plate is the longest time point for which all wells function as freshly prepared control plates.

### Potential Pitfalls and Alternative Approaches.

Poor dissolution of some antibiotics can lead to inconsistent concentrations across wells, impacting the accuracy of the concentration gradient. We test and optimize protocols for fully dissolving each drug by using gentle heating, DMSO, or pH adjustment, if necessary.

Evaluate and Refine the POCAST™ Instrument with UTI Patient Urine Samples

We evaluate and refine the POCAST™ prototype using patient urine samples with and without infection. We assess the performance of POCAST™, as well as optimize the instrumentation and fine tune the ML algorithm based on the testing and validation feedback.

After building the prototypes and testing for basic operational functionality, we first test one instrument at the ASU lab for ID/AST performance with clean urine spiked with bacteria and urine samples collected from patients with and without UTIs from Mayo Clinic. Using parameter assessments described above, we adjust and refine the prototype systems and instruments as needed to achieve benchmarks. Next, we set up a prototype in the clinical microbiology laboratory at Mayo Clinic Arizona for end user evaluation of the instrument. We evaluate susceptibility testing with patient urine samples and compare the POCAST™ results with the results obtained from reference technologies (broth microdilution). To mimic rare, but urgent, drug-resistant cases, we mainly use normal urine spiked with CRE or ESBL-producing Enterobacteriaceae for performance assessment. We focus on key performance features, including reliability, usability, robustness, and validity.

Refine the ML Model to Achieve Fast and Accurate ID/AST with POCAST™ Prototypes.

We use additional data collected with the prototype instrument to fine tune the ML models for fast and accurate ID and AST. At least 500 patient urine samples are tested. Combined with the ˜700 patient urine samples data we have collected, as well as our data collected from ˜300 pure bacterial culture and clean urine spiked with bacteria, over 1,500 samples are available for training the ML model, which is sufficient according to our power analysis. For ID, we train the ML model with datasets from cultured bacteria (model strains or isolates from patient samples; data collected from ASU), as well as non-pathogen datasets in real patient samples from Mayo Clinic. For AST, we train the model with de-identified real patient samples (collected from Mayo Clinic) using results obtained with the CLSI gold standard methods (BD Phoenix at Mayo Clinic). For all data collected, we use a common cross-validation method, such as 10-fold cross validation to evaluate the ML models. True positive (TP), true negative (TN), false positive (FP), and false negative (FN) metrics will be calculated based on prediction of the trained classifiers. Sensitivity, specificity, positive predictive value (PPV, precision), negative predictive value (NPV), and accuracy (ACC), along with the receiver operating characteristic (ROC) curves are assessed for the models. Additional features, such as category agreement and major discrepancy, are evaluated according to the FDA AST standards.

### Potential Pitfalls and Alternative Approaches.

Since the ML model recognizes phenotypic changes, rare resistant cases can be covered with trained models using antibiotics with the same mechanism (e.g., CRE and ESBL producing Enterobacteriaceae share similar β-lactam resistance traits, which will be trained with ampicillin resistance). We use verified antibiotic-resistant (e.g., CRE, ESBL E. coli) strains in pure culture or in spiked urine as an alternative dataset.

The POCAST™ system is capable of rapid (<3 hours from raw urine sample loading to results) and accurate UTI pathogen identification (Specificity: ≥90% and Sensitivity: >90%), and AST (Category Agreement: 89.9%; Major Discrepancy: 3%; and Very Major Discrepancy: 1.5%) for 8 common UTI antibiotics (FIG. 14), which meet the general acceptance criteria from the FDA.

### Example 2: Microplate-Based High Throughput Large Volume Scattering Imaging System (HT-LVSi) for Rapid Antimicrobial Susceptibility Test of Mycobacterium abscessus in Liquid Samples

The LVSim system was adapted to accommodate the sedimentation behavior of Mycobacterium abscessus smooth during drug susceptibility testing. Unlike previous imaging modalities with the LVSim using UTI bacterial species, M. abscessus exhibited clumping and sedimentation in Middlebrook 7H9 medium causing low count readings over time. To maintain visualization of the clumped bacteria, the imaging focal plane (Z-position) was shifted from the mid-solution position to the bottom of the well plate, where mycobacterial aggregates accumulated over time. No additional hardware changes were required beyond this focus adjustment, as the existing optical configuration remained adequate for backscattering detection. Imaging was conducted over a 24-hour period at 3-hour intervals to monitor morphological and drug-induced responses across four drugs: Amikacin, Bedaquiline, Clarithromycin, and Clofazamine. To minimize optical artifacts and static background interference, each well underwent frame-wise background subtraction using its initial image at timepoint 0 as a reference, followed by SIFT-based correction to align subsequent images and remove plate imperfections. Over the 24-hour imaging sequence, increased aggregate formation was observed in all M. abscessus wells during early time points, consistent with cell clumping and sedimentation; however, wells containing inhibitory antibiotic concentrations demonstrated reduced aggregate size and number, indicating drug-mediated suppression of clump formation and cell viability.

Some further aspects are defined in the following clauses:
Clause 1: A method of determining antibiotic susceptibility of bacteria, the method comprising: obtaining a series of images from partitioned samples over a length of time using a large volume scattering imaging (LVSi) technique to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and, outputting the set of predicted identity and antibiotic susceptibility data, thereby determining the antibiotic susceptibility of the bacteria.
Clause 2: The method of Clause 1, wherein the obtaining step is performed in the absence of culture-based isolation and/or enrichment of the partitioned samples.
Clause 3: The method of Clause 1 or Clause 2, comprising substantially removing non-bacterial particles and non-bacterial cells from the partitioned samples prior to performing the obtaining step.
Clause 4: The method of any one of the preceding Clauses 1-3, wherein the partitioned samples comprise between about 103 bacterial cells/mL and about 108 bacterial cells/mL.
Clause 5: The method of any one of the preceding Clauses 1-4, wherein the partitioned samples comprise urine.
Clause 6: The method of any one of the preceding Clauses 1-5, wherein the partitioned samples are disposed in a microplate.
Clause 7: The method of any one of the preceding Clauses 1-6, wherein the multiple phenotypic features and/or the phenotypic feature changes are selected from the group consisting of: a cell count, a cellular shape, a cell division, a cellular motion, a cellular physiology, a cell size, and a cellular morphology.
Clause 8: The method of any one of the preceding Clauses 1-7, wherein the multiple phenotypic features comprise 3, 4, 5, 6, 7, 8, 9, 10, or more phenotypic features.
Clause 9: The method of any one of the preceding Clauses 1-8, wherein the set of image data comprises LVSi videos.
Clause 10: The method of any one of the preceding Clauses 1-9, further comprising administering an antibiotic to treat a bacterial infection in a test subject based at least in part on the set of predicted identity and antibiotic susceptibility data.
Clause 11: The method of any one of the preceding Clauses 1-10, comprising determining minimum inhibition concentrations (MIC) of the antibiotics.
Clause 12: The method of any one of the preceding Clauses 1-11, wherein the bacteria comprise one or more of Escherichia coli, Klebsiella pneumoniae, Staphylococcus saprophyticus, or Enterococcus faecalis.
Clause 13: A kit for performing the method of any one of the preceding Clauses 1-12.
Clause 14: A point-of-care device for performing the method of any one of the preceding Clauses 1-13.
Clause 15: The method of any one of the preceding Clauses 1-14, comprising obtaining the partitioned samples from one or more test subjects.
Clause 16: The method of any one of the preceding Clauses 1-15, comprising outputting the set of predicted identity and antibiotic susceptibility data within about 3 hours of obtaining the partitioned samples from one or more test subjects.
Clause 17: The method of any one of the preceding Clauses 1-16, wherein the test subjects are suspected of having urinary tract infections (UTIs).
Clause 18: A system for determining antibiotic susceptibility of bacteria, comprising: a large volume scattering imaging (LVSi) apparatus configured to receive partitioned samples; and, a controller operably connected to the LVSi apparatus, which controller comprises, or is capable of accessing, computer readable media comprising non-transitory computer-executable instructions which, when executed by at least one electronic processor, perform at least: obtaining a series of images from partitioned samples over a length of time using LVSi apparatus to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and, outputting the set of predicted identity and antibiotic susceptibility data.
Clause 19: The system of Clause 18, wherein the set of image data comprises LVSi videos.
Clause 20: A computer readable media comprising non-transitory computer executable instruction which, when executed by at least electronic processor, perform at least: obtaining a series of images from partitioned samples over a length of time using a large volume scattering imaging (LVSi) technique to produce a set of image data, wherein at least some of the partitioned samples comprise one or more bacterial cells and one or more antibiotics; extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features; passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and, outputting the set of predicted identity and antibiotic susceptibility data.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A method of determining antibiotic susceptibility of bacteria, the method comprising:
obtaining a series of images from partitioned samples over a length of time using a large volume scattering imaging (LVSi) technique to produce a set of image data, wherein at least some of the partitioned samples comprise one or more bacterial cells and one or more antibiotics;
extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features;
passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and,
outputting the set of predicted identity and antibiotic susceptibility data, thereby determining the antibiotic susceptibility of the bacteria.

2. The method of claim 1, wherein the obtaining step is performed in the absence of culture-based isolation and/or enrichment of the partitioned samples.

3. The method of claim 1, comprising substantially removing non-bacterial particles and non-bacterial cells from the partitioned samples prior to performing the obtaining step.

4. The method of claim 1, wherein the partitioned samples comprise between about 10³ bacterial cells/mL and about 10⁸ bacterial cells/mL.

5. The method of claim 1, wherein the partitioned samples comprise urine.

6. The method of claim 1, wherein the partitioned samples are disposed in a microplate.

7. The method of claim 1, wherein the multiple phenotypic features and/or the phenotypic feature changes are selected from the group consisting of: a cell count, a cellular shape, a cell division, a cellular motion, a cellular physiology, a cell size, and a cellular morphology.

8. The method of claim 1, wherein the multiple phenotypic features comprise 3, 4, 5, 6, 7, 8, 9, 10, or more phenotypic features.

9. The method of claim 1, wherein the set of image data comprises LVSi videos.

10. The method of claim 1, further comprising administering an antibiotic to treat a bacterial infection in a test subject based at least in part on the set of predicted identity and antibiotic susceptibility data.

11. The method of claim 1, comprising determining minimum inhibition concentrations (MIC) of the antibiotics.

12. The method of claim 1, wherein the bacteria comprise one or more of Escherichia coli, Klebsiella pneumoniae, Staphylococcus saprophyticus, or Enterococcus faecalis.

13. A kit for performing the method of claim 1.

14. A point-of-care device for performing the method of claim 1.

15. The method of claim 1, comprising obtaining the partitioned samples from one or more test subjects.

16. The method of claim 15, comprising outputting the set of predicted identity and antibiotic susceptibility data within about 3 hours of obtaining the partitioned samples from one or more test subjects.

17. The method of claim 15, wherein the test subjects are suspected of having urinary tract infections (UTIs).

18. A system for determining antibiotic susceptibility of bacteria, comprising:
a large volume scattering imaging (LVSi) apparatus configured to receive partitioned samples; and,
a controller operably connected to the LVSi apparatus, which controller comprises, or is capable of accessing, computer readable media comprising non-transitory computer-executable instructions which, when executed by at least one electronic processor, perform at least:
obtaining a series of images from partitioned samples over a length of time using LVSi apparatus to produce a set of image data, wherein at least some of the partitioned samples comprise one or more bacterial cells and one or more antibiotics;
extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features;
passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and,
outputting the set of predicted identity and antibiotic susceptibility data.

19. The system of claim 18, wherein the set of image data comprises LVSi videos.

20. A computer readable media comprising non-transitory computer executable instruction which, when executed by at least electronic processor, perform at least:
obtaining a series of images from partitioned samples over a length of time using a large volume scattering imaging (LVSi) technique to produce a set of image data, wherein at least some of the partitioned samples comprise one or more bacterial cells and one or more antibiotics;
extracting multiple phenotypic features from individual bacterial cells in the partitioned samples in parallel with one another and detecting one or more phenotypic feature changes associated with bacterial growth and response to the antibiotics in the set of image data over a selected period of time to produce a set of extracted phenotypic features;
passing the set of extracted phenotypic features through a trained machine learning model that predicts the identity and the antibiotic susceptibility of the bacterial cells in the partitioned samples to produce a set of predicted identity and antibiotic susceptibility data, wherein the machine learning model is trained using a series of images obtained from a plurality of reference samples that comprise ground truth results; and,
outputting the set of predicted identity and antibiotic susceptibility data.
